(19) United States
(12) Patent Application Publication
(10) Pub. No.: US 2003/0199948 A1
(43) Pub. Date: Oct. 23, 2003

(54) Multiport neurological screening cable

(76) Inventors: Scott B. Kokones, Minneapolis, MN (US); Jane Lambie Kohnen, Minneapolis, MN (US); Luis E. Fesser, Woodbury, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/125,857

(22) Filed: Apr. 19, 2002

(51) Int. Cl. A61N 1/05

(52) U.S. Cl. 607/117

(57) ABSTRACT

A neurological stimulation screening cable is disclosed to improve physician flexibility during the screening procedure for neurological conditions such as pain, movement disorders, functional disorders. The screening cable comprises a proximal screening connector, at least two screening conductors, a distal screening connector configured for receiving at least two implantable neurological leads, at least two lead receptors each having at least two receptor contacts, and a cover having a cover open position for permitting access to the lead receptors and a cover closed position for enclosing the lead receptors.

### FIELD OF THE INVENTION

This disclosure relates to a medical device and more particularly to implantable neurological electrical stimulators and implantable electrical stimulation leads.

### BACKGROUND OF THE INVENTION

The medical device industry produces a wide variety of electronic and mechanical devices for treating patient medical conditions such as pacemakers, defibrillators, neuro-stimulators and therapeutic substance delivery pumps. Medical devices can be configured to be surgically implanted or connected externally to the patient receiving treatment. Clinicians use medical devices alone or in combination with therapeutic substance therapies and surgery to treat patient medical conditions. For some medical conditions, medical devices provide the best and sometimes the only therapy to restore an individual to a more healthful condition and a fuller life. One type of medical device is an implantable neurological stimulation system that can be used to treat conditions such as pain, movement disorders, pelvic floor disorders, gastroparesis, and a wide variety of other medical conditions. The neurostimulation system typically includes a neurostimulator, a stimulation lead, and an extension such as shown in Medtronic, Inc. brochure “Implantable Neurostimulation System” (1998). More specifically, the neurostimulator system can be an Itrel II® Model 7424 or an Itrel 3® Model 7425 available from Medtronic, Inc. in Minneapolis, Minn. that can be used to treat conditions such as pain, movement disorders and pelvic floor disorders. The neurostimulator is typically connected to a stimulation lead that has one or more electrodes to deliver electrical stimulation to a specific location in the patient's body.

The lead and stylet combination are part of the implantable neurostimulation system. The neuro stimulation lead is placed in the desired location of the body. The stylet wire and handle combination are used to give the lead stiffness during implantation and to aid in maneuvering the lead into the desired position. Currently several types of lead and stylet combinations exist for this purpose. An example of a stylet that engages the proximal end of the lead connector region in specified locations is the Pisces Quad Lead Model 3487A stylet available from Medtronic, Inc., Minneapolis, Minn., USA. Once the lead is believed to be placed in the appropriate position within the body the lead, with or without the stylet, is coupled with a neurostimulation screening cable, which is connected to a neuro stimulation screening device. The screening device can be programmed to send different combinations, strengths and frequencies of electrical stimulation to the patient. The screening cable provides a connection to, and electrical pathway between the stimulation lead or percutaneous extension and the neuro stimulation screening device. The patient is questioned to determine if the stimulation covers the desired region of the body. Provided results are favorable the patient receives a temporary implant of the stimulation lead system. Either the stimulation lead or percutaneous extension is attached to the screening cable for a trial screening period so the patient can assess the efficacy of the system in normal life settings. Patient is sent home with an external neurostimulator that sends electrical stimulation to the stimulation lead via the screening cable during the trial period. This trial period can range from 1 to 30 days depending on the physician and the country in which the trial occurs. This trial period is used to access the efficacy of the stimulation therapy for the patient.

The neuro stimulation field is growing rapidly and with growth comes new therapies and ways of using existing products. Currently neurostimulation therapies primarily use one lead at a time. A market exists and will continue to grow where multiple leads and multiple types of leads could be used simultaneously. Currently a screening cable does not exist that would allow multiple lead and multiple lead type screening. It could be cumbersome and uncomfortable for a patient to carry multiple screening devices. There is, therefore, a need for a screening cable system that can accommodate multiple lead types, as well as multiple lead arrays to give physicians the flexibility to treat each patient in the best possible way while still making the screening procedure as easy and comfortable as possible for the patient.

Thus, embodiments of the improved neuro stimulation screening cable are disclosed that improve physician flexibility during the screening procedure.

### BRIEF SUMMARY OF THE INVENTION

A multiport neurological screening cable that improves physician flexibility during the screening procedure comprises a proximal screening connector, at least two screening conductors, and a distal screening connector. The two screening conductors are electrically connected to the proximal screening connector. The distal screening connector is configured for receiving at least two implantable neurological leads, and the distal screening connector is electrically connected to the screening conductors. The distal screening connector includes a housing, at least two lead receptors each having at least two receptor contacts carried in the housing, and a cover. The cover is coupled to the housing and has a cover open position for permitting access to the lead receptors and a cover closed position for enclosing the lead receptors.

### BRIEF DESCRIPTION OF THE DRAWINGS

FIG. 1 shows a general environmental view for a neurological system embodiment;

FIG. 2 shows a neurological system embodiment;

FIG. 3 shows a neurological lead embodiment;

FIG. 4 shows another neurological lead embodiment;

FIG. 5 shows a percutaneous extension embodiment;

FIG. 6 shows a screening cable embodiment;

FIG. 7 shows a screening cable with an open cover embodiment;

FIG. 8 shows an exploded view of a screening cable embodiment;

FIG. 9 shows a stimulation lead with stylet, a percutaneous extension, and an opened screening cable embodiment;

FIG. 10 shows two different stimulation leads and a screening cable with closed cover embodiment;

FIG. 11 shows cover switch and housing cover in open position embodiment;

FIG. 12 shows cover switch and housing cover in closed position embodiment; and,

FIG. 13 shows a flow diagram of a method for connecting an implantable neurological lead to an external neurostimulator embodiment.

### DETAILED DESCRIPTION OF THE INVENTION

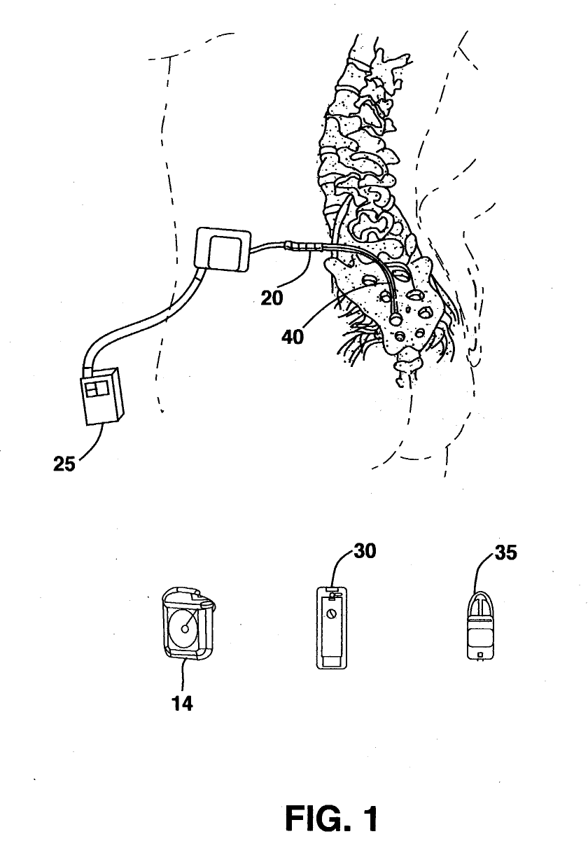

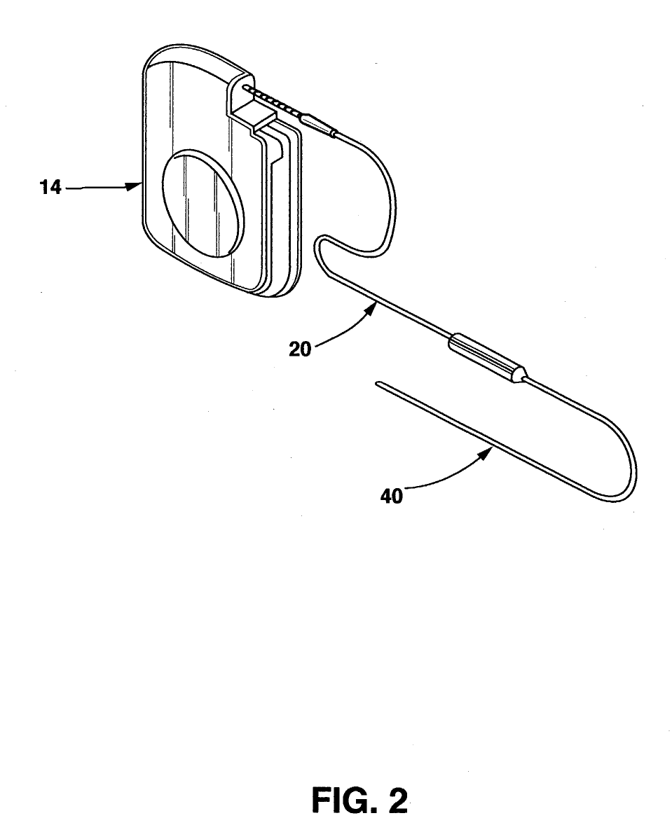

FIG. 1 shows the general environment of an Implantable Neuro Stimulator (INS) medical device 14. The neurological system generally includes an INS 14 FIG. 2, a neurological lead 40, a lead extension 20, an External Neuro Stimulator (ENS) 25, a physician programmer 30, and a patient programmer 35. The INS 14 can be a Medtronic Itrel II® Model 7424 or an Itrel 3® Model 7425 or the like, both of which are commercially available.

The neurological lead 40 is a small medical wire with special insulation. The neurological lead 40 includes one or more insulated electrical conductors with a connector on the proximal end and electrical contacts on the distal end. Some neurological leads are designed to be inserted into a patient percutaneously, such as the Model 3487A Pisces-Quad® lead available from Medtronic, Inc. of Minneapolis Minn., and some neurological leads are designed to be surgically implanted, such as the Model 3998 Specify® lead also available from Medtronic. The neurological lead 40 may also be a paddle having a plurality of electrodes including, for example, a Medtronic paddle having model number 3587A. In yet another embodiment, the neurological lead 40 may provide electrical stimulation as well as drug infusion. Those skilled in the art will appreciate that any variety of neurological leads 40 may be used to practice the present invention.

The neurological lead 40 is implanted and positioned to stimulate a specific site in the spinal cord or the brain. Alternatively, the neurological lead 40 may be positioned along a peripheral nerve or adjacent neural tissue ganglia like the sympathetic chain or it may be positioned to stimulate muscle tissue. The neurological lead 40 contains one or more electrodes (small electrical contacts) through which electrical stimulation is delivered from the ENS 25 or INS 14 to the targeted neural tissue. If the spinal cord is to be stimulated, the neurological lead 40 may have electrodes that are epidural, intrathecal or placed into the spinal cord itself. Effective spinal cord stimulation may be achieved by any of these lead placements.

Although the lead connector can be connected directly to the ENS 25 or INS 14, typically the lead connector is connected to a lead extension 20 which can be either temporary for use with an ENS 25 or permanent for use with an INS 14. An example of the lead extension 20 is Model 7495 available from Medtronic.

The ENS 25 functions similarly to the INS 14 but is not designed for implantation. The ENS 25 is used to test the efficacy of stimulation therapy for the patient before the INS 14 is surgically implanted. An example of an ENS 25 is a Model 3625 Screener available from Medtronic. The ENS 25 is used in conjunction with a screening cable 70 which accepts the stimulation lead 40 or lead extension 20 and creates an electrical pathway to the ENS 25.

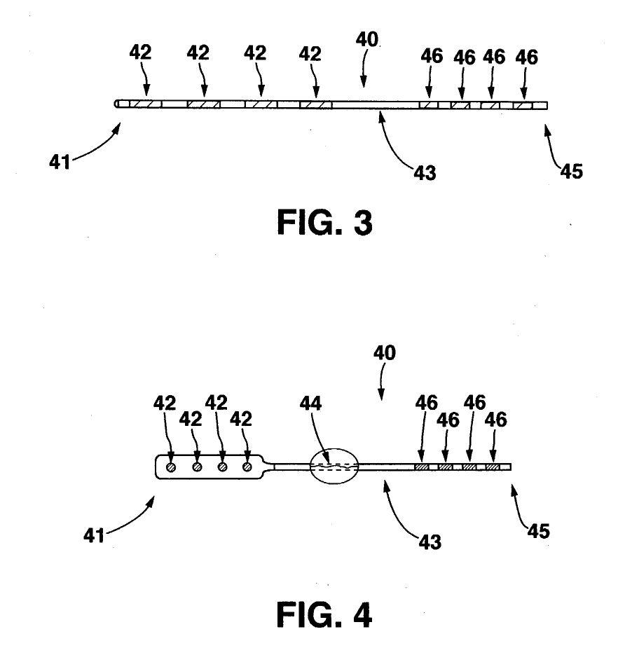

FIG. 3 shows a percutaneous neurological lead 40 and FIG. 4 shows a surgical paddle neurological lead 40. The neurological lead 40 can be configured as a neurological lead, a neurological sensing lead, and a combination of both as a neurological stimulation and sensing lead. The implantable neurological lead 40 comprises a lead body 43, at least one conductor 44, at least one electrical connector 46, at least one electrode 42, and a stylet 50. The lead body 43 has a body proximal end 45, and a body distal end 41. The lead body 43 can be composed of a wide variety of materials and configurations. Materials can include, but not be limited to silicone rubber, polyurethane, fluoropolymers and the like. Configurations could include monolumen and multilumen tubings.

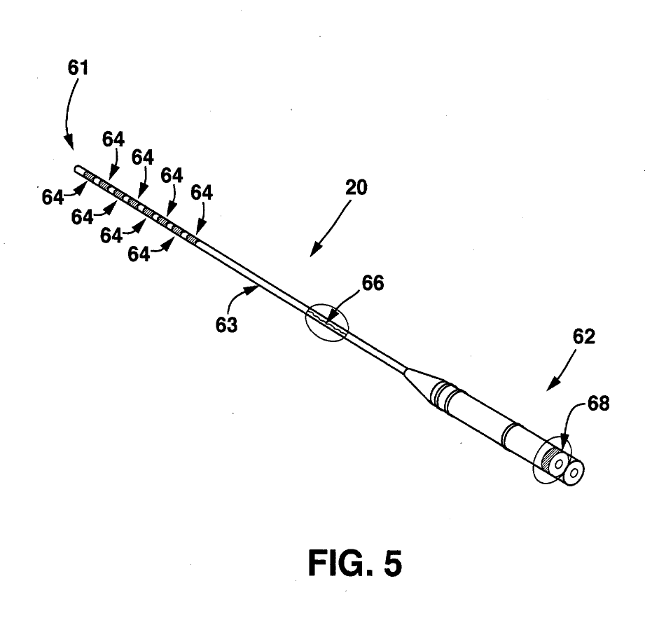

FIG. 5 shows an extension 20. The extension 20 comprises an extension body 63, at least one electrical connector 64, at least one lead connector 68, and at least one extension conductor 66. The extension body 63 has a body proximal end 61 and a body distal end 62. The extension body 63 can be composed of a wide variety of materials and configurations. Materials can include, but not be limited to, silicone rubber, polyurethane, fluoropolymers and the like. Configurations could include monolumen and multilumen tubings.

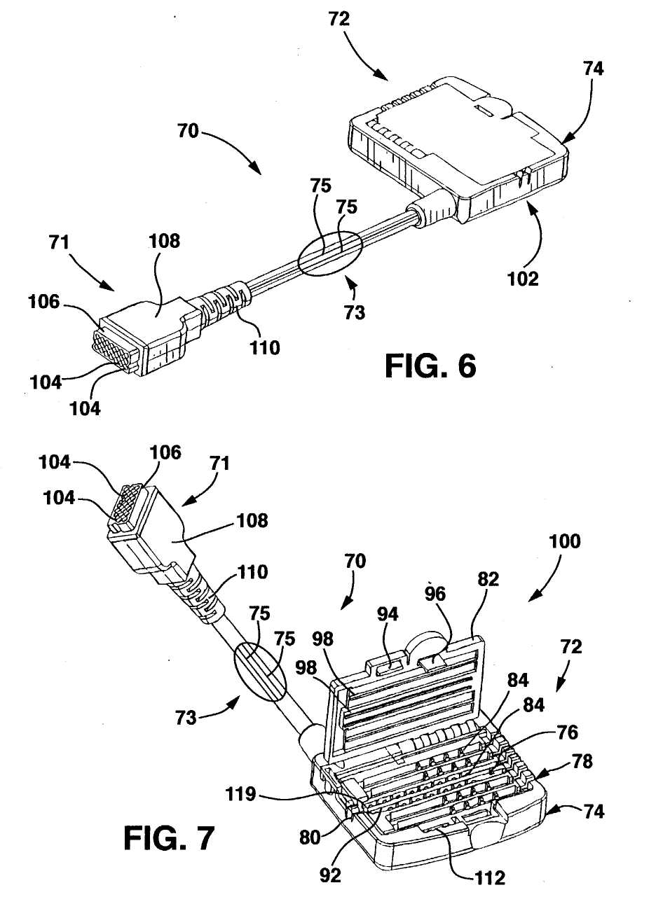

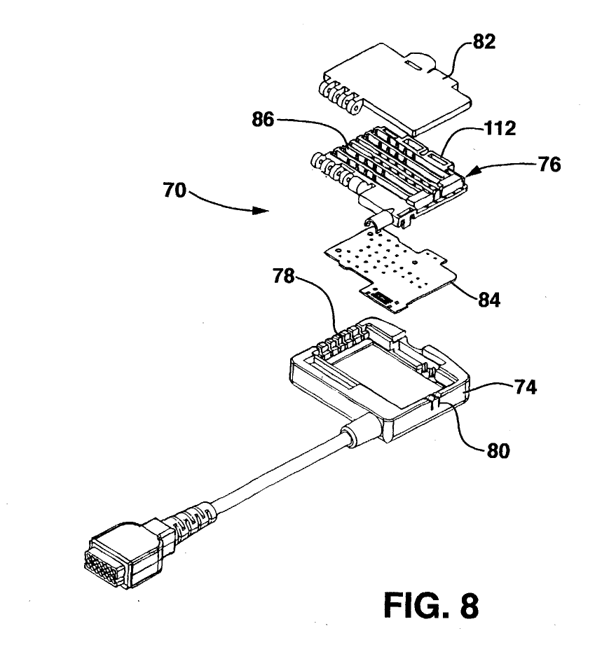

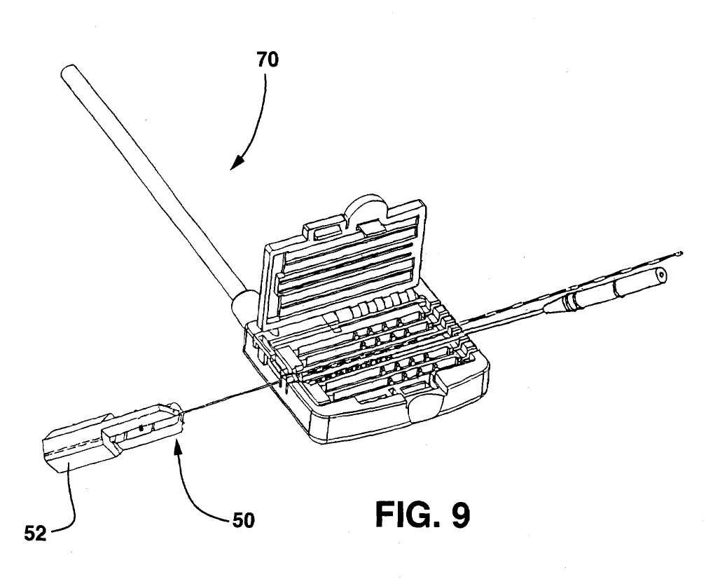

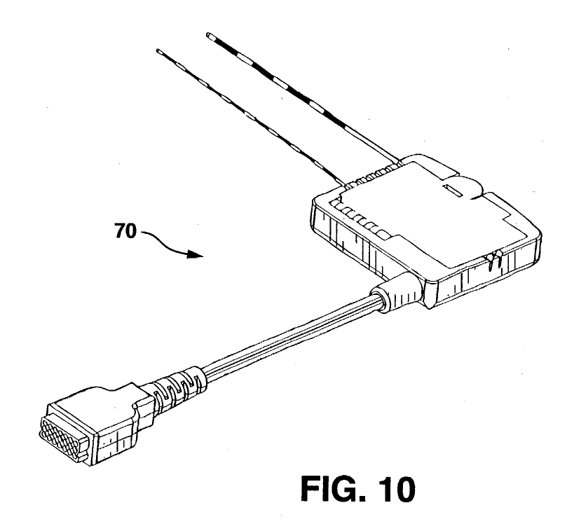

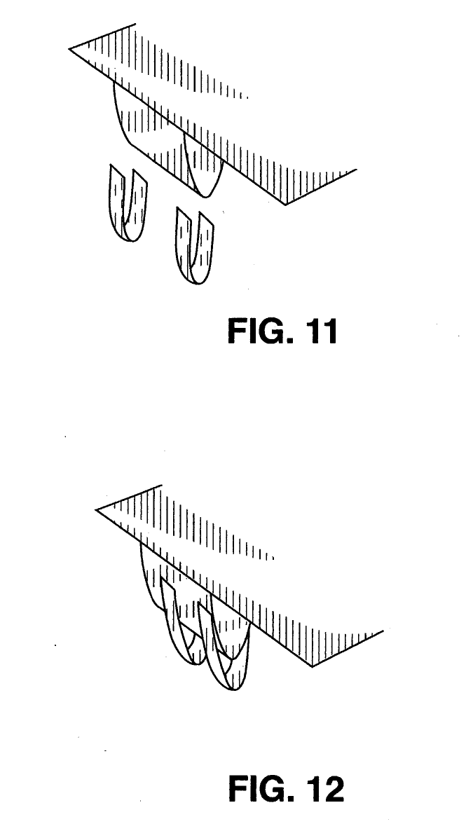

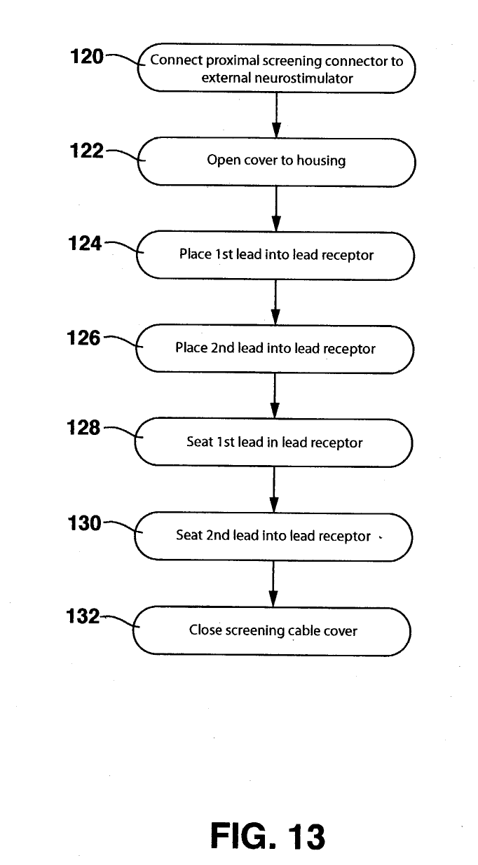

FIGS. 6-12 show various views and embodiments of the implantable neurological lead 40 with multiport neurological screening cable 70. The multiport neurological screening cable 70 includes a proximal screening connector 71, at least two screening conductors 75, and a distal screening connector 72. The proximal screening connector 71 consists of a at least two contact pins 104 used to make electrical contact with the external neurostimulator 25, a pin housing 106 which holds and configures the contact pins 104 and keys for insertion into the external neurostimulator 25, and a proximal connector overmold 108 which mechanically connects the screening cable body 73 to the pin housing 106 and creates a strain relief 110 for added flex life.

The at least two screening conductors 75 are electrically connected to the proximal screening connector 71. The two screening conductors 75 are contained in the screening cable body 73 extending from the proximal screening connector 71 to the distal screening connector 72. The screening conductors 75 can be manufactured from materials such as tinseled wire bare copper wire, silver wire, and the like. Screening conductor 75 configurations could include stranded, braided or solid wire configurations. Each screening conductor 75 is individually insulated with a polymer. The polymer could be, but is not limited to a thermoplastic elastomer. Other materials that act as electrical insulators can be used. The screening cable body 73 which contains the screening conductors 75 can also be constructed from a polymer could be, but is not limited to a thermoplastic elastomer. The pin connector 104 carried on the proximal screening connector 71 electrically connects to the screening conductor 75. The pin connector 104 in combination with the pin housing 106 is configured to couple with the external neurostimulator 25. The pc board 84 is carried in the distal screening connector 72 and electrically connected to the screening conductors 75. The receptor contacts 88, which are carried on the housing 76 are electrically connected to the pc board 84. The receptor contacts 88 accept the neurological lead 40 and makes electrical contact with the electrical connector 46. The aforementioned pathway delivers electrical stimulation from an external neurostimulator 25 to the stimulation lead 40.

The distal screening connector 72 is configured for receiving at least two implantable neurological leads 40. The distal screening connector 72 is electrically connected to the screening conductors 75 and includes an overmold 74 a housing 76, a pc board 84, at least two lead receptors 86, and a cover 82. The at least two lead receptors 86 are configured for receiving a first implantable neurological lead 40 and a second implantable neurological lead 40 that can be a wide variety of neurological leads. The neurological leads 40 can be embodiments such as a four-conductor neurological lead, a four-conductor extension, a four-conductor neurological lead with stylet handle, a four conductor percutaneous extension with stylet handle, an eight-conductor neurological lead, an eight-conductor extension, an eight-conductor neurological lead with stylet handle, and the like.

The housing 76 contained in the distal screening connector 72 is configured for receiving at least two implantable neurological leads 40. The housing 76 includes at least two lead receptors 86, and a cover switch housing 112. The overmold 74 contains at least two lead entry channels 78, and at least one stylet channel 80. The overmold 74 surrounds and mechanically couples to the premold 76, and a pc board 84. The overmold 74 consists of large radiused edges to aide in patient comfort. The overmold 74 mechanically connects the distal screening connector 72 to the screening cable body 73. Materials for the overmold 74 can include, but is not limited to a thermoplastic elastomer such as santoprene. The stylet channel 80 provides the ability for the stylet handle 52 to be decoupled and partially removed from the stimulation lead 40 permitting screening while the stylet 50 is in the neurological lead 40. The lead entry channels 78 provide a space for the lead to exit the screening cable 70. Materials for the housing 76 can include but are not limited to ultem, polycarbonate, polysulfone, and other hard plastics.

The at least two lead receptors 86 are carried by the housing 76. The at least two lead receptors 86 are of varying sizes or shapes. The lead receptors 86 are designed to accept a variety of different neurological lead 40 types for neurological lead stimulation or sensing. Lead types can include but are not limited to quadripolar leads with or without the use of a stylet 50 or connector and octapolar leads with or without the use of a stylet 50 or connector. Each lead receptor 86 houses at least one receptor contact 88, which is used to make electrical contact with a neurological lead 40. Material for the receptor contact 88 can include but is not limited to gold plated beryllium copper. The lead receptors 86 may or may not contain grippers 92 used to grip the lead proximal end 45 to prevent the stimulation lead 40 from moving during trial stimulation. The multiple lead receptors 86 in the screening cable housing 76 can be, but do not have to be occupied by a stimulation lead during screening.

The cover 82 is coupled to the housing 76. The cover 82 having a cover open position 100 for permitting access to the lead receptors 86 and a cover closed position 102 for enclosing the lead receptors 86. The housing 76 is mechanically connected to a cover 82 via a hinge. The cover 82 consists of a cover switch 94, a closing latch 96 and at least two lead cover details 98. The cover 82 has two positions, a screening cable open position 100 and a screening cable closed position 102. The cover 82 contains at least two types of lead details 98 that are used for simultaneously forcing multiple types of neurological leads 40 into the receptor contacts 88 that are contained in the lead receptors 86 while in the cover closed position 102. The cover 82 can be constructed from but is not limited to ultem, polycarbonate, polysulfone and other hard plastic materials.

Some embodiments of the screening cable 70 can include a disconnect feature by which the external neurostimulator 25 cannot send electrical stimulation to the screening cable receptor contacts 88 until the distal screening connector 72 is in the cable closed position 102. Electrical stimulation can only be sent through the screening conductors 75 when the proximal screening connector 71 is coupled with the external neurostimulator 25 and the distal screening connector 72 is in the cable closed position 102. When the screening cable 70 is in the cable closed position 102 the cover switch 94 on the cover 82 makes electrical contact with two of the screening cable receptor contacts 88 in the cover housing switch 112 creating a complete circuit. This electrical signal is returned to the external neurostimulator 25 activating the ability to send stimulation therapy to the other screening cable receptor contacts 88.

FIG. 12 shows a flowchart of a method for inserting a neurological lead 40 into the improved neurological screening cable 70. The method begins by connecting 120 the proximal screening connector 71 to the external neurostimulator 25. Continue by opening 122 the cover 82 to the distal screening connector 72 thus putting the distal screening connector 72 in the cover open position 100. This allows access to the first and second lead receptors 86. Once the cover 82 is opened, remove stylet handle 50 from the lead body proximal end 45 of the first neurological 40, when applicable. Some neurological leads 40 may be inserted into the screening cable 70 while coupled with a stylet handle 50 or connector and some neurological leads 40 may couple with the screening cable 70 directly. The procedure for both options will follow the same steps for insertion into the screening cable 70. Place 124 the first neurological lead 40 into the first lead receptor 86 by pushing the lead body proximal end 45 against the lead stop 119 in the first lead receptor 86. Making sure the electrical connectors 46 of the lead 40 are aligned with the screening cable receptor contacts 88. Place 126 the second neurological lead 40 into the second lead receptor 86 by pushing the lead body proximal end 45 against the lead stop 119 in the second lead receptor 86. Making sure the electrical connectors 46 of the lead 40 are aligned with the screening cable receptor contacts 88. Seat 128 the first neurological lead 40 into the receptor contacts 88 contained in the first lead receptor 86 by pressing the first neurological lead 40 with a thumb or forefinger into the receptor contacts 88. Seat 130 the second neurological lead 40 into the receptor contacts 88 contained in the second lead receptor 86 by pressing the second neurological lead 40 with a thumb or forefinger into the receptor contacts 88. Once both leads 40 are seated in the lead receptors 86 and receptor contacts 88, close 132 the cover 82 thus putting the distal screening connector 72 into the cover closed position 102 denying access to the lead receptors 86. By putting the distal screening connector 72 in the cover closed position 102 the cover details 98 further reinforce the seating of both the first and second neurological leads 40. Furthermore, the process of closing the cover 82 can act as the sole mechanism for seating all neurological leads 40 if using a thumb or forefinger in the previous two steps does not properly seat the neurological leads 40. By closing the cover 82, the cover switch 94 is also put in the switch closed position 102 allowing electrical stimulation to be sent to the receptor contacts 88.

Thus, embodiments of the multiport neurological screening cable are disclosed to improve physician flexibility during the screening procedure. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A multiport neurological screening cable, comprising:
a proximal screening connector;
at least two screening conductors electrically connected to the proximal screening connector; and,
a distal screening connector configured for receiving at least two implantable neurological leads, the distal screening connector being electrically connected to the screening conductors and including,
a housing,
at least two lead receptors carried by the housing, the lead receptors each having at least one receptor contact.
a cover coupled to the housing, the cover having a cover open position for permitting access to the lead receptors and a cover closed position for enclosing the lead receptors.

2. The multiport neurological screening cable as in claim 1 wherein the lead receptors comprise a first lead receptor for accepting a first implantable neurological lead and a second lead receptor for accepting a second implantable neurological lead.

3. The multiport neurological screening cable as in claim 1 further comprising a cover switch having a switch housing detail and a switch cover detail, the cover switch having an open position to disable the screening cable and a closed position to enable the screening cable.

4. The multiport neurological screening cable as in claim 1 further comprising at least two lead openings formed in the housing for receiving implantable neurological leads into the lead receptors.

5. The multiport neurological screening cable as in claim 1 further comprising at least one stylet opening formed in the housing to permit at least one stylet to extend outside the housing.

6. The multiport neurological screening cable as in claim 1 further comprising cover details configured to seat the neurological leads into the receptor contact.

7. The multiport neurological screening cable as in claim 1 further comprising a cover lock operated by the cover to secure the cover to the housing.

8. The multiport neurological screening cable as in claim 1 further comprising an overmold that surrounds a portion of the housing and mechanically couples to the housing.

9. A multiport neurological screening cable, comprising:
a proximal screening connector;
at least two screening conductors electrically connected to the proximal screening connector; and,
a distal screening connector configured for receiving at least two implantable neurological leads, the distal screening connector being electrically connected to the screening conductors and including,
a housing,
means for receiving at least two neurological leads, the means for receiving carried by the housing; and,
means for covering coupled to the housing, the means for covering selectively permitting access to the lead receptors and denying access to the lead receptors.

10. A multiport distal screening connector, comprising:
a housing,
at least two lead receptors carried by the housing configured for receiving at least two implantable neurological leads, the lead receptors each having at least one receptor contact; and,
a cover coupled to the housing, the cover having a cover open position for permitting access to the lead receptors and a cover closed position for denying access to the lead receptors.

11. The multiport distal screening connector as in claim 10 further comprising a cover switch having a switch housing detail and a switch cover detail, the cover switch having an open position to disable the screening cable and a closed position to enable the screening cable.

12. A method for connecting an implantable neurological lead to an external neurostimulator, comprising:
connecting a proximal screening connector to an external neurostimulator;
opening a cover to a housing;
placing a first implantable neurological lead into first lead receptor carried in the housing;
placing a second implantable neurological lead into a second lead receptor carried in the housing;
seating the first implantable neurological lead into the first lead receptor to electrically couple the first implantable neurological lead to a first receptor contact; and,
seating the second implantable neurological lead into the second lead receptor to electrically couple the second implantable neurological lead to a second receptor contact; and,
closing the cover over the first implantable neurological lead and the second neurological lead.

13. The method as in claim 12, further comprising activating a cover switch to enable the screening cable creating electrical continuity between the external neurostimulator and the first neurological lead and the second neurological lead.
